(19) United States
(12) Patent Application Publication
(10) Pub. No.: US 2011/0288366 A1
(43) Pub. Date: Nov. 24, 2011

(54) Surgical Approach to Treat Coronary Artery Syndromes

(76) Inventor: Mounir E. Nassar, Pittsford, NY (US)

(21) Appl. No.: 13/111,388

(22) Filed: May 19, 2011

Related U.S. Application Data
(60) Provisional application No. 61/346,058, filed on May 19, 2010.

(51) Int. Cl. A61N 1/362

(52) U.S. Cl. 600/16

(57) ABSTRACT

A treatment for congestive heart failure due to acute or chronic coronary syndromes with myocardial infarction comprising excising from the patient's heart an auricular appendage and grafting the auricular appendage to the damaged heart muscle.

### BACKGROUND OF THE INVENTION

The present invention relates to cardiac surgery, and more particularly relates to a surgical technique of using an auricular appendage graft to augment areas of weakened heart muscle.

Congestive heart failure occurs when the heart fails to deliver a sufficient output of blood to meet the metabolic requirements of the organs and tissues of the body. Its manifestations include a lower blood pressure resulting in poor perfusion of tissues which may result in conditions such as dyspnea, orthopnea, and nocturnal paroxysmal dyspnea secondary to lung fluid congestion-edema, high jugular venous pressure, renal insufficiency, and peripheral pitting edema. A type of congestive heart failure includes systolic dysfunction due to impaired myocardial contraction leading to left ventricular failure. There are many causes of congestive heart failure including hypertension, acute and chronic coronary artery syndromes and which are the most common causes. Low ejection fraction below 40.

Another type of congestive heart failure is diastolic dysfunction manifesting in non- pliable, stiff left ventricle resulting in low blood pressure with normal ejection fraction and leading to left ventricular failure. Both types of congestive heart failure described above may occur simultaneously in the same patient.

### SUMMARY OF THE INVENTION

The present invention describes and claims a treatment for congestive heart failure due to acute or chronic coronary syndromes with myocardial infarction or non ST on the electrocardiogram myocardial infarction myocardial infarction or prior coronary artery bypass graft, or percutaneous coronary stent failure all resulting in congestive heart failure. The inventive technique involves surgically transplanting the right or left auricular appendage of the heart to the damaged weakened area of the diseased myocardium. [It is important to preserve the venae cavae and the sinoatrial node on the right and pulmonary veins on the left, depending which auricular appendage is used]. A team of cardiothoracic surgeon(s) and cardiologist(s) are required. Cardiac echoes and cardiac output will be measured pre and post-op and followed up with chest x-ray, electrocardiogram, and cardiac echoes with physical therapy for several months. This procedure may be perfected in experiments on animals, dogs, cats, sheep etc.

### BRIEF DESCRIPTION OF THE DRAWING

FIG. 1 A is a view of a human heart (as redrawn from Gray's Anatomy textbook) showing the auricular appendages;

FIG. 1B is another view of the human heart showing labeled parts thereof (source: www.facebook.cominote.php?note id=177071863900) ;

FIG. 2 is a view of the heart showing a blocked left anterior descending artery with infarction of muscle area of the heart (source: http://www.philadelphia-reflections.com/images/heart_attack.jpg);

FIG. 3 is a representation of a Coronary artery bypass graft operation to treat myocardial infarction with a saphenous vein graft from aorta to a point distal coronary obstruction (source: http://www.surgery.ucla.edukardiac/images/saphenouslg.jpg);

FIG. 4 is a representation of a Coronary bypass graft from left subclavian artery using internal mammary artery to a point distal to coronary artery obstruction site (source: http://www.surgery.ucla.edu/cardiac/images/saphenouslg.jpg).

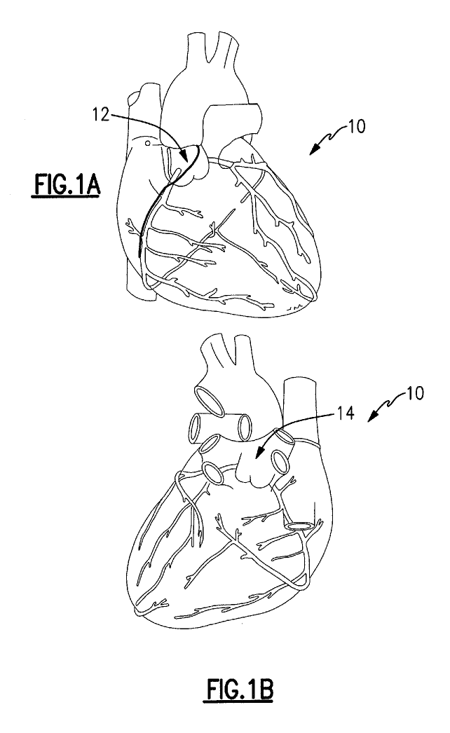

### DETAILED DESCRIPTION

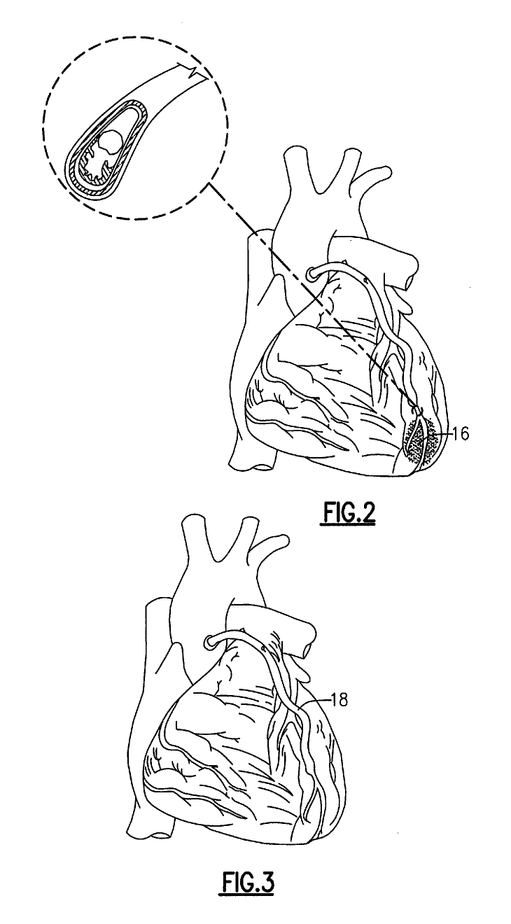

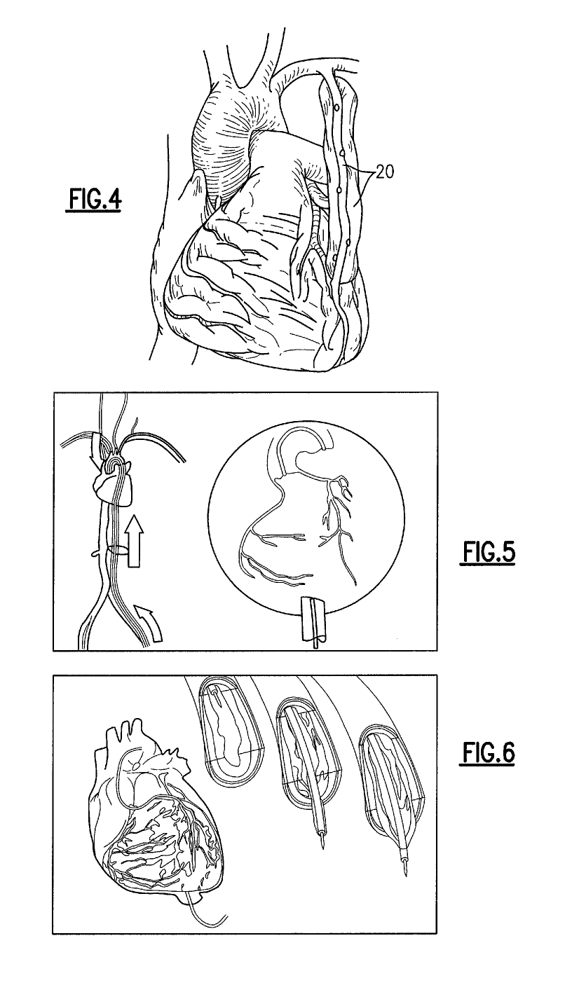

Referring now to the drawing, there is seen in the Figures a human heart which includes right and left auricle appendages 12 and 14 which are extensions of the main right and left atrium, respectively. FIG. 2 is a view of the heart showing a blocked left anterior descending artery with infarction of muscle area of the heart as indicated at reference numeral 16. Current treatments for myocardial infarctions include those seen in FIGS. 3-6, for example. FIG. 3 shows a coronary artery bypass graft operation to treat myocardial infarction with a saphenous vein graft 18, and FIG. 4 shows a graft 20 using the internal mammary artery from subclavical to left coronary artery branch distal to the obstruction site. If the bypass graft segment gets blocked in the future, the patient may have a second heart attack or, if the blockage is gradual, the area of cardiac muscle supplied by the graft may start to fail to contract well and heart failure may ensue. A balloon angioplasty may be inserted into blocked coronary artery and inflated to relieve obstruction in the coronary artery. A stent can then be inserted to keep vessel open although stents are also known to occasionally fail.

According to the present invention, either of the auricular appendages 12 and 14 are excised and transplanted onto the affected area 16 in the manner of a graft. Since the auricular appendages are muscular tissue, once the graft has taken hold the auricular tissue will assist the cardiac muscle in this area to restore normal pumping action to the heart. It is expected that the transplanted auricular appendage will take as a graft and provide collateral circulation to the areas affected and restore blood flow and also strengthen the damaged myocardial wall.

1. A method of treating congestive heart failure due to acute or chronic coronary syndromes wherein the patient's heart muscle has been damaged, said method comprising the steps of:
a) surgically excising an auricular appendage from the patient's heart; and
b) grafting the excised auricular appendage to the location of the patient's damaged heart muscle.
